(19) United States
(12) Patent Application Publication
(10) Pub. No.: US 2024/0274297 A1
(43) Pub. Date: Aug. 15, 2024

(54) SURGICAL WARNING

(71) Applicant: Aiomic ApS, Copenhagen N (DK)

(72) Inventors: Martin Hylleholt Sillesen, Frederiksberg (DK); Alexander Bonde, Frederiksberg (DK)

(21) Appl. No.: 18/569,643

(22) PCT Filed: Jul. 15, 2022
(86) PCT No.: PCT/EP2022/069869

§ 371 Date: Dec. 13, 2023

(30) Foreign Application Priority Data
Jul. 15, 2021 (EP) 21185879.0

(51) Int. Cl. G16H 50/30; G16H 10/60

(52) U.S. Cl. CPC G16H 50/30; G16H 10/60

(57) ABSTRACT

Disclosed are systems and methods for continuously detecting, and optionally classifying, abnormal perfusion patterns in tissue by means of fluorescence imaging. One embodiment relates to a computer implemented method for detecting (and/or identifying) one or more areas having an abnormal perfusion pattern in tissue of a subject, for example during a medical procedure, the method including: continuously acquiring fluorescence images of the tissue, wherein the fluorescence images are associated with a fluorescent output signal correlated with an input signal defined by a series of boluses of at least one fluorescent imaging agent, and wherein the series of boluses is administered with a predefined and/or controlled duration between subsequent boluses, analyzing the fluorescence images, identifying at least one tissue area with normal perfusion, defining a normal perfusion pattern (in an intensity domain and) in a time domain, and detecting, in the fluorescence images, possible tissue areas with abnormal (non-normal) perfusion pattern.

### FIELD OF INVENTION

The present disclosure relates to methods and systems for predicting an outcome of a complication of a patient and for further training a trained machine learning model; and, for locally training a generally trained machine learning model.

### BACKGROUND OF THE INVENTION

Each year approximately 234 million major surgical procedures are performed, worldwide. While approximately 4% of patients undergoing a surgical procedure die as a result of a surgery, around 15% of patients experience a prolonged recovery due to postoperative complications (PCs).

PCs arising after a surgical procedure within first months is a massive burden on hospital resources as well as on individual patients. In addition to the obvious impact of PCs on the individual patient, PCs increase treatment costs between 119-172%, compared to the uncomplicated recoveries. For example in the US, PCs cost to the US healthcare system is an additional amount of 31 billion USD annually in treatment costs (1). In Denmark, approx. 1.5 million surgical procedures are performed annually. Although the incidence and cost of PCs have not been adequately described, an extrapolation of data indicates that more than 225,000 Danes can possibly be struck by a PC each year, resulting in an added cost for the health care system of approximately 787 million USD annually, corresponding to 2.7% of the total health care expenditures.

Due to rising health-care costs and constrained financial resources, recognizing at-risk patients has a great importance. Generally, majority of PCs are potentially preventable if early warnings are provided. Currently, the ability to predict PCs prior to a surgery, is based on standardized protocols for all patients. Thus, the current approach where patients are given prophylactic treatments according to Standard Operating Procedures SOPs (e.g., thromboprophylaxis and antibiotics), fails to provide a significant reduction in PCs. Furthermore, the current approach for predicting PCs prior to surgery subject to variations depending on the individual assessment of each doctor or physician assessing the patient resulting in a subjective prediction of PCs. The terms doctor and physician can be understood to be interchangeable.

An alternative approach for receiving early warnings is to deliver a strategy, wherein a wide range of data associated with the patient may allow predicting the likely of development of a disease, and survivability of the patient. However, the current technology supported by trained models are based on registry data, which may not transfer well to individual and/or local patients. Furthermore, tracking the incidence of each PCs in real-time is currently not possible.

Thus, there is a great demand for a technological solution for an improved approach for predicting development of complications after a surgery for each patient.

### SUMMARY OF THE INVENTION

In general, the disclosure concerns predicting an outcome of a complication of a patient. With the present disclosure, a complication can be tracked in real-time after a surgery. If/when patient's data is updated after a surgery, so the prediction of an outcome of a complication can be updated. Such an approach can allow for taking pre-emptive measures before the surgery as well as during or after the surgery. Said approach can also open up the possibilities for training of a machine learning model continuously and locally.

It is therefore an object of the present disclosure to provide methods and systems for predicting risks of complications after a surgery.

The present inventor has realised that prediction of complication, such as postoperative complications (PCs) does not have to be limited to a database, wherein the predictions are standardized for all patients.

It is therefore a further object of the presently disclosed approach to enhance reliability of predicting a true outcome of complications based on data associated with the patient before, during or after the surgery. This solution should allow a personalized outcome prediction before, during or after the surgery.

It is a further object of the present disclosure to provide a personalized outcome prediction based on data associated with a dynamically trained machine-learning model. The present inventor has further realised that the trained dataset used in current machine learning models are based on a static dataset, e.g., trained on a dataset, which is based on received health data of the patient prior to a surgery.

Thus, it is a further object of the present disclosure to re-train the trained machine-learning model successively as data comes in, such that an outcome of a complication, such as developing a PC, can be predicted throughout the treatment.

It is yet a further object of the present disclosure to train a generally trained machine learning model locally. This can be achieved by primarily training a model on a general cohort representing multiple different surgical patients from across different healthcare sites, in order to capture the general relationships between risk factors and outcomes. Once the model primarily trained, the model can, by means of the concept of transfer learning, be retrained on data from a local hospital or surgical unit. Consequently, a risk prediction relevant for the specific surgical patients can be provided. This implies that the re-training will adjust factors including, but not limited to weights in a deep learning network thereby better adapting to the local settings.

The present disclosure therefore relates, in a first aspect, to a method for predicting an outcome of a complication of a patient and for further training a trained machine-learning model, wherein the method comprises the steps of
receiving a medical report of a patient,
analysing said medical report for an outcome of a complication of the patient by a trained machine learning model, such as a convolutional neural network model, thereby obtaining a complication outcome;
receiving a true outcome of the complication,
updating the trained machine learning model to become a locally trained machine learning model.

In general, the disclosed method relates to predicting an outcome of a complication of a patient and for further training a trained machine-learning model. After receiving a medical report of a patient, comprising health data of the patient or any other type of patient data, said medical report is analysed for receiving an outcome of a complication, such as risk of developing a PC after a surgery. Additionally, a true outcome of the complication, for example true outcome of whether or not a PC is developed, can be used for updating the trained machine-learning model so that a reliability of the predicting an outcome of a complication can be enhanced.

Thus, an advantage of the presently disclosed approach is that previously trained machine-learning model can be retrained by a true outcome after a surgery in real-time. This also implies that the machine-learning model is configured such that the previously trained machine-learning model can be updated in real-time. Because the machine-learning model can be updated on the basis of the true outcome of the complication and the medical report of the patient having the true outcome, from one aspect, the presently disclosed approach provides hospitals with an improved risk drives of complications.

A further advantage of the presently disclosed approach is that the complication outcome can be personalised. Because the presently disclosed approach proposes receiving a medical report of a patient, comprising health data of the patient or any other type of patient data, before, during or after the surgery. Thus, said medical report can be analysed for assessing a risk of developing a complication before, during or after the surgery. This foresees that the machine-learning model can be updated on the basis of patient data, wherein the data refers to medical report data before, during or after the surgery. Because the medical report of a patient can be updated after a surgery, reanalysing the updated medical report of the patient may provide an updated outcome of the complication, wherein the updated complication outcome can be within a certain predetermined time.

Additionally, the proposed approach can automatically track and report the true outcome of the complications and update the trained machine learning model accordingly. This leads to a more reliable and objective approach for predicting an outcome of a complication compared to the static input and outcome dependencies present in the current technology.

A further advantage of the presently disclosed method is that the machine learning model can be updated locally in local hospitals, cottage hospitals, health care institutions or any other institutions where a surgery takes place. Furthermore, the trained machine-learning model can be trained with data from a specific department.

Because the method provides an update of the machine learning model locally and dynamically with the true outcome of the complication and the patient medical report, the reliability of the outcome of a complication predicted by the trained machine learning model can be enhanced.

Additionally, retraining the trained machine learning model with the true outcome of the complication of the local patient improves the complication outcome, after a number of surgical procedures have been analysed. An important aspect of the presently disclosed approach is therefore to allow the machine learning model to learn local hospital unit features over time, as the standard operating procedures for each hospitals and departments can be independent from each other.

It should be noted that, some doctors/hospitals may use one drug or one treatment and other doctors/hospitals may use another drug or another treatment. Depending on drug/treatment that is used in one hospital may influence the outcome, which the AI system will pick up, such that if a hospital using a drug/treatment that leads to more complications, the AI model will pick up the data related to said drug/treatment, which will influence the outcome. The locally trained machine learning model for that hospital will predict an outcome of a complication of a patient based on the drug(s) or treatment(s) used at that hospital. It will even be possible to use differently locally trained machine learning model to compare, which drug(s) or treatment(s) is/are the best.

For example, the situation in a small hospital can be different compared to the situation in the large hospital. In the large hospital, there are many doctors, who are able to specialise within a certain subdiscipline of their medical specialty. In the small hospital, there are few doctors, who will need a broad knowledge and therefore are not able to specialize to the same extent. There may be rare sicknesses that the small hospital is not able to recognise/treat. The local update of the model will be able to take care of such differences. Because such an approach would provide altering the weighting to attain the influence of an input on an output, it may be desirable to increase the weighting locally. Advantageously, the impact can be implicated faster for the local update. Thus, the local updates can be weighted with a weight greater than 1, advantageously greater than 1.5.

Yet another advantage of the present disclosure is to provide operational efficiency. Physicians and patients may benefit from the proposed procedure for assessing the risk of developing a complication, taking precautions, counselling and decision-making. Furthermore, more objective predictions for predicting complications prior to a surgery can be provided. As a result, with the proposed approach, the operational costs, hospital resources, burden on physicians and patients can be decreased significantly.

The local training of the machine learning model can be that data of a medical report of a patient is fed into the machine learning model when the patient is admitted to the hospital preferably including the date of the admittance. Every time a doctor is examining the patient, the results and/or conclusions of the examination can be added to the machine learning model preferably including the date of the examination. The results and/or conclusions of the examination added to the machine learning model can comprise or can be the complication outcome. In that way the complication outcome can vary with time and can be updated many times. The complication outcome can e.g. be high/low blood pressure, high/low heat beat, abnormal blood values, headache, dizziness, nausea, formations of a blood clot, stroke, infarction, internal haemorrhages e.g. in the brain, death, etc. or lack of any complication.

In an embodiment, the method may comprise the steps of receiving a second medical report of a second patient at the same location as the patient, analysing said second medical report for a second outcome of a second complication of the second patient by the locally trained machine learning model, thereby obtaining a second complication outcome, receiving a true outcome of the second complication, and updating the locally trained machine learning model to become a further locally trained machine learning model.

The advantages of this embodiment are the same as mentioned above. The same location can mean that the patient and the second patient are treated by the same physician, by the same group of physicians, preferably specialist physicians, of one single hospital. The same location can additionally mean that the patient and the second patient are treated by the same group of physicians, preferably specialist physicians, of one single group of hospitals, if the physicians of said same group of physicians work interchangeably at the different hospitals of the single group of hospitals.

The trained machine learning model is generally trained without any consideration to any location, while the locally trained machine learning model and the further locally trained machine learning model are trained locally, where the patient and the second patient have the same location.

Patients used for the trained machine learning model will not have the same location as the patient and the second patient, at least will not the majority of or all the patients used for the trained machine learning model have the same location as the patient and the second patient.

The second medical report, the second patient, the second outcome, the second complication, the second complication outcome, and the second true outcome can be a plurality of second medical reports, second patients, second outcomes, second complications, second complication outcomes, and second true outcomes, respectively, where the locally trained machine learning model is updated by each of the second medical reports, second outcomes, second complications, second complication outcomes, and second true outcomes to become the further locally trained machine learning model.

The locally trained machine learning model will be more and more local as the locally trained machine learning model develops into the further locally trained machine learning model with the advantage that the second complication outcome predicted by the further locally trained machine learning model will be closer and closer to the corresponding second true outcome.

In an embodiment, the trained machine learning model and the locally trained machine learning model can be updated dynamically to become the locally trained machine learning model and the further locally trained machine learning model, respectively.

That the machine-learning model is trained dynamically can mean that the training of a machine learning model is done continuously, like before, during, and/or after every surgery, every doctor's appointment, every time the doctor is examining the patient, and/or every month/week/day.

In an embodiment, the trained machine learning model and the locally trained machine learning model can be updated dynamically by updating before and/or during and/or after an operation for predicting the outcome or the second outcome.

The operation can be understood to mean a surgery and/or another medical operation performed by a doctor on a patient, like e.g. administering a drug to the patient and/or treating the patient and/or measuring or estimating a patient related quantity like e.g. blood pressure, pulse rate, kind of tumor, size of tumor, location of tumor, content of a body fluid, concentration or presence of a molecule in a body fluid, etc.

Finally, the present disclosure relates to a data processing system configured to perform the steps of the any of the methods described herein and a computer program comprising instructions, which, when the program is executed by a computer, cause the computer to carry out any of the methods described herein.

Consequently, the computer program and/or the system can be adapted for lowering health-care costs in the surgical environment by lowering patient morbidity and mortality, and by providing a rapid and an automatic prediction.

### DESCRIPTION OF THE DRAWINGS

The present disclosure will in the following be described in greater detail with reference to the accompanying drawings:

FIG. 1 shows one embodiment of a surgical warning system and a flowchart for predicting a risk of developing a complication.

FIG. 2 is one embodiment of an overview of data flow for predicting the risk of developing deep venous thrombosis during 30 days after a surgical procedure.

FIG. 3 shows one embodiment of an overview of data flow for training a machine learning model locally.

FIG. 4 shows one embodiment of an automated surgical complication tracking and reporting system overview.

### DETAILED DESCRIPTION OF THE INVENTION

### Medical Report

As used herein, the term medical report refers to a medical record data of a patient.

In an embodiment, the medical report of a patient can comprise health data of the patient or any other type of patient data. In a further embodiment, the medical report can comprise pre-surgical data of the patient, such as diagnoses, medication, blood sample. The medical report of the patient can further comprise patient's medical history, treatment plans, immunization dates, allergies, radiology images, and laboratory and test results. Medical report can also comprise vital signs, personal statistics like age and weight.

Preferably, the medical report can be an electronic medical record data or electronic health record data, such that the electronic medical record can be created and managed in a digital format capable of being shared, processed and analysed, for example by a data processing system.

An advantage of the electronic medical report data may be that a large medical report database such that medical record data of a plurality of patients and demographics can be combined. Medical report data of one or more patients can also mobilize to a platform such as a cloud service or an internal server wherein medical report data can train and/or retrain a machine learning model.

Furthermore, said medical report data can be available instantly to various platforms, such as healthcare personals, authorized users, computer programs. This implies that the medical report of a patient can be accessed in real-time, analysed and updated with further data. Alternatively, the medical report data of a patient can be configured for capturing the state of the patient across time.

### Complication Outcome

After a procedure or a treatment, the patient may experience one or more complications. Preferably, the medical report data can be analysed to identify the patients having a complication after a surgery.

In an embodiment, the presently disclosed approach comprises the step of analysing said medical report for an outcome of a complication of the patient. In a further embodiment, the complication can be a sickness or a surgery. Thus, the step of analysing the medical report may be for an outcome of a surgery. The step of analysing the medical report may also be for an outcome of a sickness. Said complication may for example be thromboembolic, infectious and organ specific complications, such as sepsis, bleeding, ventilator support, intubation, embolism, vascular accident, shock, and so on. Alternatively, the complication may be mortality.

Preferably, the analysis of the said medical report can be performed for one or more complication specified prior to the analysis. This implies that, the step of analysis of the medical report of the patient may be for an outcome of a specified complication. In an advantageous embodiment, the outcome of the complication may comprise a risk level of the complication. Specifically, predicting a risk level of a complication between 0 and 100%, 0% being the lowest risk level for experiencing said complication after a surgery, may be desirable. Because the risk level of developing complications following surgical procedures can be assessed prior to the surgery, necessary precautions can be taken in advance.

In an embodiment, the complication outcome may be the outcome within a certain predetermined time. A patient having experienced one or more complications within a certain predetermined time after a surgical procedure can be tracked for analysing the health report of said patient and predicting the complication outcome of the patient during the certain predetermined time after the surgical procedure. In a further embodiment, the certain predetermined time can be calculated from the step of analyzing said medical report or from a surgery. Preferably, the certain predetermined time may be 3 days, 1 week, 2 weeks, 4 weeks, 1 year or more after a surgical procedure. More preferably, the certain predetermined time may be 30 days after a surgery.

### Machine Learning Model

An advantage of the presently disclosed approach may be that from one aspect, the present approach relates to an automated surgical complication tracking and reporting system by means of an artificial intelligence model. Preferably, the artificial intelligence model is a machine-learning model.

In an embodiment, a trained machine learning model can analyse the medical report of a patient for an outcome of a complication of said patient. The trained machine learning model can be configured such that patients having experienced a surgery and a complication after a surgery can be classified. Then, when the machine learning model receives data related to the health report of a patient, the machine learning model can analyse the data for an outcome of a complication. Additionally, a risk-level of experiencing a complication can be predicted.

In an embodiment, the trained machine learning model can be a convolutional neural network model. Alternatively, the trained machine learning model can be a deep neural network. The machine learning model can be trained to predict risks of a complication arising within a certain predetermined time after a surgical procedure. In terms of surgical risk predictions, several predictive modelling technique can be adapted. Thus, in an embodiment the trained machine learning model can comprise a logistic regression model, entity embeddings and/or a random forest model.

An advantage of the deep neural networks is to incorporate high-dimensional input variables by unbiased investigations into the driving factors behind complications, while factoring in the often-non-linear associations between inputs and dependent variables. Dependent variables used in predictive modelling can for example be complications such as mortality and a plurality of PCs. Input variables can be chosen among data provided within medical report of the patient, such as data related to patient's medical history, treatment plans, immunization dates, allergies, radiology images, and laboratory and test results, vital signs, personal statistics, and so on.

Trained machine learning model can be built on number of input variables. However, in order to find a reasonable trade-off between the amount of input variables and model performance, it can be advantageous to train one or more models having different complexity in terms of number of input variables. For example, a first model can be trained with between 15-20 input variables, a second model can be trained with between 40-50 input variables and a third model can be trained with between 80-90 input variables.

Furthermore, the predictive modelling can comprise input variables related to a blood sample of the patient. In an embodiment, the step of analysing said medical report by the trained machine learning model provides information about whether a blood sample of the patient should be obtained, such as genotyping and complex biomarker analysis. Alternatively, the blood sample may be a DNA sample.

This implies that a dependent variable may relate to whether a DNA sample of the patient should be obtained or not. Data related to DNA sample can be deployed depending on a risk level of said dependent variable. Alternatively, the data related to DNA sample can only be deployed depending on a risk level of a specific complication. Coupling of a request for DNA sampling with a risk level prediction can minimize expensive and cumbersome procedure of augmenting the DNA data by complex biomarkers from any patients at any time.

### Dynamic Training

An important aspect of the presently disclosed approach is the ability to leverage real-time data from medical report for predictive modelling. From one aspect, complications can be tracked and reported in real-time, such that a complication outcome of a tracked patient can be received from medical professionals and a true complication outcome can be diagnosed in the said tracked patient. This implies that, a large surgical dataset which incorporates medical report data with true complication outcome of patients can be modelled.

The present approach can therefore receive, assess and update a medical report of a patient, comprising health data of the patient or any other type of patient data, before, during or after the surgery such that the outcome of the complication can be updated. In an embodiment, the method comprises the step of updating the machine learning model before, during or after the surgery. The updating of the machine learning model dynamically is therefore based on the patient data, wherein the data can refer to medical report data before, during or after the surgery. Consequently, with the present dynamic training, a risk of developing a complication can be assessed before, during or after the surgery.

In an embodiment, the presently disclosed approach can comprise a step of reanalysing the medical report for an updated outcome of the complication of the patient by the trained machine learning model. In a preferred embodiment, the step of reanalysing is performed after the medical report has been updated by an update selected from the group of
medication changes,
anaesthesia data,
X-ray data,
lab results such as ECG data (cardiac cardiograms), blood sample data, and microbiological culture data.
Vital signs such as pulse and blood pressure,
Fluid input and output data,
Data related to insertion and removal of drains, and intravenous access,
Procedural data, such as operations, physiotherapy, wound care.

In an embodiment, the step of reanalysing the medical report is performed periodically, such as at least once a week, preferably at least once a day. Advantageously, when a surgical procedure for a tracked patient was booked, the medical report data can be reanalysed to update the outcome of the complication. Thus, risk prediction can be updated, which then can be used by caregivers as a decision support tool.

Reanalysis of the medical report for updating the outcome of the complication of the patient can be performed periodically and continuously until a predetermined time period is achieved.

An important aspect of the presently disclosed approach is that, the medical report data of the patient can be monitored postoperatively for a predetermined time; for example for 4 weeks from the surgical procedure, an outcome tracker can track and register complications. Based on registered complication data, the medical report data can be updated. Thus, the registered complications can be used for retraining of the machine learning model and thereby allowing for continuous updating of the model, and allowing the model to adapt to changes in treatment practices and patient demographics over time.

Preferably, the steps of analysing medical report for an updated outcome of the complication of the patient by the trained machine learning model and updating of the machine learning model frequently with the true outcome of a complication can be performed periodically for the patient, preferably for 4 weeks after the surgery. The concept of analysing an updated medical report of the patient for an updated outcome of a complication while updating the machine learning model accordingly can be regarded as a dynamic training.

Thus, it is possible to predict risk of developing complications following surgical procedures by means of a machine learning model such that the machine learning model can be retrained dynamically, wherein the model can be updated continuously and periodically at least with a true outcome of the complication.

A further advantage of the dynamic retraining of the machine learning model can be to enhance prediction models for predicting the dependent variables wherein the input variables can be periodically updated. From one aspect, hereby-disclosed approach can enable accessing and mobilizing medical report data in real time between the medical report database on which the initial trained model is based on and a secure cloud service or an internal server, and establishing a method for executing a real-time machine learning model.

In an embodiment, the trained machine learning model is a long short-term neural network model. Preferably, dynamic retraining of the trained machine learning model is based on a long short-term neural network, a convolutional neural network or a combination thereof.

The trained machine learning model can be trained on a large dataset of patient medical report, wherein each report includes medically relevant pre- , intra- and post-operative parameters. Specifically, pre- , intra- and post-operative parameters may be input variables for trained machine learning model. Preferably, the trained machine learning model can be configured such that the model can be retrained to output a complication occurring for any given patient within a certain predetermined time window after a surgery based on input variables. Thus, the machine learning model can be configured such that the risk prediction model can run to process at least pre-, intra-operative parameters and to predict likely complications.

There can be lags of unknown duration between important events, such as between true outcome of a complication in a time period after a surgery. Thus, long short-term neural network models can be advantageous as long short-term neural network models can provide feedback connections, wherein multiple data points and sequences of data can be processed based on time series data, thereby suiting to classify, process and make predictions on time series data, e.g., during the time period after a surgery.

### Local Training

Furthermore, the presently disclosed approach relates to a method for training a generally trained machine learning model locally. In an embodiment, the generally trained machine learning model is trained by providing the generally trained machine learning model with many inputs, I, and many corresponding outputs, O. Preferably, many inputs may be input data based on medical report data and many outputs may be dependent variables. Many outputs therefore may be an outcome of a complication. The generally trained machine learning model may be trained generally such that the inputs and outputs may be related to many patients having experienced a surgery at many departments, hospitals, regions.

In a further embodiment, the generally trained machine learning model can be trained by providing further inputs and corresponding further outputs locally.

In an embodiment, the (further) locally trained machine learning model is a first (further) locally trained machine learning model at a first location, wherein the method further comprises step of updating the trained machine learning model at a second location different from the first location to become a second (further) locally trained machine learning model.

In a preferred embodiment, the method for training a generally trained machine learning model locally may comprise the step of receiving a medical report of a local patient, and analysing said medical report for an outcome of a complication of the local patient by the trained machine learning model, such as a convolutional neural network model, thereby obtaining a complication outcome.

Because pre-, intra-, and post-operative parameters may be input variables for trained machine learning model, a true outcome of a complication of a local patient can be updated as input variable. Thus, the method for training a generally trained machine learning model locally may further comprise the step of receiving a true outcome of the complication of the local patient and updating the trained machine learning model with the true outcome of the complication of the local patient.

In a further embodiment, first (further) locally trained machine learning model and the second (further) locally trained machine learning model are compared, and wherein medical reports and true outcomes of the first and second (further) locally trained machine learning models are compared for determining an optimal treatment/drug of a certain disease.

An advantage of adapting a dynamic retraining of a trained machine learning model, is that local data from individual hospitals or individual units can allow gradual learning of local features. As a result of feeding standardized patient data to the individual machine learning models of individual units or hospitals, it may be possible to analyse outcome differences between different units and/or hospitals, for example when factoring in the underlying patient disease spectrum. Furthermore, the local training may enhance identification of features when/if outcomes differ between units.

Identification of features between units and hospital can promote to identify whether a feature is patient-centered or treatment-centered.

### System

In an embodiment, a data processing system can comprise a processor configured to perform the steps of the method for predicting an outcome of a complication of a patient and for further training a trained machine learning model and/or for locally training a generally trained machine learning model. In a further embodiment, a processor can be a processing device.

In another embodiment, a computer program can comprise instructions, which, when the program is executed by a computer, cause the computer to carry out the steps of the any of the methods as descried herein. Preferably, the computer program is executable on a processing device. Thus, methods as described herein may be a computer-implemented method such that disclosed methods can be carried out by a computer.

The computer may be a data processing system. The data processing system may comprise a processor and a memory, wherein the memory may comprise a computer-readable storage media. The computer-readable storage media may be a non-transitory computer-readable storage media. The computer-readable storage media may store a computer program instructions, which are executable by the processor. Thus, the memory can be accessible to the processor and can hold the instructions of the program to execute. Preferably, the computer program can store instructions for causing the processor to perform steps of any of the methods as described herein.

Additionally, singular terms, such as “memory”, “computer-readable storage media,” or “processor” may refer to a plurality of memories and/or computer-readable storage medias and/or processors.

Finally, the disclosed approach provides methods and systems at least for hospital decision makers on a society-wide scale. Furthermore, the presently disclosed approach can be adapted to other clinical settings wherein an automated registry and tracking could be of value; e.g., hospital acquired infections.

### EXAMPLES

### Complication Outcome

From one aspect, the presently disclosed approach is configured for tracking and predicting one or more compilations. Complications may for example be referred as PCs. Table 1 shows 18 PCs and the definitions of each PCs. While the presently disclosed approach focuses on hereby-referred 18 PCs, number of PCs and respective definitions can be subject to change.

| | Variable | Definition |
|---|---|---|
| Output | Mortality | Death during the intraoperative |
| features | | period or within 30 days |
| | | after the primary procedure |
| | Superficial | Infection that involves |
| | incisional | only skin or subcutaneous |
| | surgical site | tissue of the surgical incision |
| | infection | |
| | Deep incisional | Infection that involves tissue |
| | surgical | beneath skin and subcutaneous fat, |
| | site infection | such as fascial and muscle layers |
| | Organ/space | Infection that involves any |
| | surgical | part other than the incision, |
| | site infection | which was opened or |
| | | manipulated during the operation |
| | Wound disruption | The spontaneous reopening |
| | | of a previously |
| | | surgically closed wound |
| | Pneumonia | Infection of one or both lungs |
| | Unplanned | The placement of a breathing |
| | intubation | tube with ventilator support |
| | Pulmonary | Lodging of a blood clot in |
| | embolism | the pulmonary artery with |
| | | subsequent obstruction of blood |
| | | supply to the lung parenchyma |
| | On ventilator >48 h | Cumulative ventilator support, |
| | | exceeding 48 hours within 30 days |
| | | after the primary procedure |
| | Progressive renal | A rise in creatinine of >2 mg/dl |
| | insufficiency | from preoperative value, but |
| | | with no requirement dialysis |
| | Acute renal failure | Acute Renal Failure Requiring |
| | | Dialysis in a patient who did not |
| | | require dialysis preoperatively |
| | Urinary tract | An infection in the urinary |
| | infection | tract (kidneys, ureters, |
| | | bladder, and urethra) |
| | Stroke/cerebral | Motor, sensory, or cognitive |
| | vascular accident | dysfunction which persists for |
| | | 24 hours or more in the setting of a |
| | | suspected stroke |
| | Cardiac arrest | The absence of cardiac rhythm |
| | requiring | or presence of a chaotic cardiac |
| | cardiopulmonary | rhythm requiring the initiation of |
| | resuscitation | cardiopulmonary resuscitation |
| | Myocardial | Documentation of ECG changes |
| | infarction | or elevated troponin |
| | | indicative of acute MI or |
| | | Physician, PA or NP diagnosis |
| | | of myocardial infarction |
| | Deep vein | New diagnosis of blood clot |
| | thrombosis/ | or thrombus within the |
| | thrombophlebitis | venous system requiring therapy |
| | Sepsis | The systemic response to infection. |
| | Septic shock | Sepsis associated with organ and/or |
| | | circulatory dysfunction. |
| | Bleeding requiring | Transfusion of red blood cells, |
| | transfusions | whole blood, autologous blood |
| | | or cell-saver products. |

Initially, a dataset comprising a plurality of medical report data of a plurality of patients can be analysed such that the medical report data of each individual is coupled with an above-described PC that each individual has experienced. Consequently, a classified surgical PC registry can be created. A complication outcome of a random sample of patients, such as 50 patients, among the plurality of patients, each with and/or without having the above-described 18 PCs can be extracted from said dataset, for the purpose of validating the PC registry based on the above-described 18 PCs. This validation step will require manual curation of data by healthcare trained staff capable of classifying PC's from reviews of health care data. Identification of relevant genomic single nucleotide polymorphisms (SNPs) associated with PCs will be achieved through analyses of large national biobanks with coupled registries detailing surgically relevant outcomes.

Thus, a trained machine learning model comprising a real-time risk prediction model for above-described 18 PCs can be based on said dataset comprising the plurality of medical report data of the plurality of patients. One example of training a machine learning model for predicting postoperative surgical complications is disclosed by Bonde et al. [1].

The classification of PCs based on the medical report of patients can be used to train a machine learning model. The trained machine learning model can be retrained such that a risks of patients developing a PC, among above-described 18 PCs, after a surgery can be predicted in real-time and periodically based on medical report data of the patient and other clinical registries such as medication changes, anaesthesia data, X-ray data, lab results and so on. An advantage of the real-time, periodic risk prediction model can be that the risk of developing a PC can be analysed more accurately, because while some PCs can be a subset of another PC; e.g., wound infections, other PCs may be directly related to diagnostic data and standard blood sample data; e.g., acute renal failure.

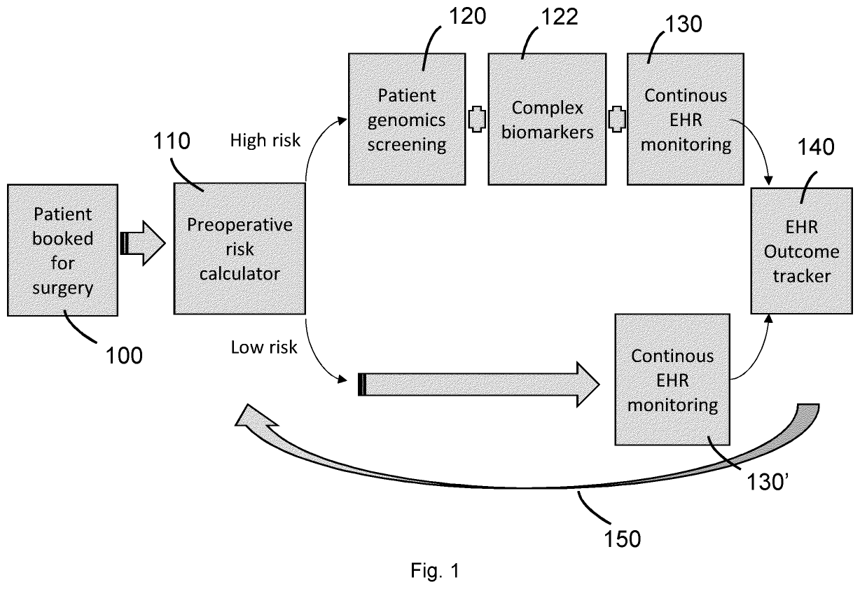

FIG. 1 is an embodiment of an overview of a Surgical Advanced Warning (SAW) system, for example, a data processing system configured to perform the steps of the any of the methods as described herein, for predicting a risk of developing a complication. FIG. 1 can also regarded as an exemplary method for predicting a risk of developing a complication.

Upon scheduling a patient for a surgery in step 100, a tracker is attached to the medical report of the patient. Medical report of the patient can for example be an Electronic Health Record (EHR) data. The tracker indicates an active surveillance of the EHR data. Thus, the tracker can be configured for monitoring EHR data in steps 130 and 120′ and further identifying and classifying in step 140 whether one or more PCs among above-described 18 PCs occurred during a given patient admission. The tracker can also track a combination of EHR data and other clinical registries such as medication changes, anaesthesia data, X-ray data, and laboratory results and so on.

SAW system is configured such that the data acquired from the EHR data can be fed to a secure cloud service. The data acquired from the EHR data may for example be one or more input variables and can be fed to the machine learning model. After analysing the EHR data of the patient based on one or more input variables, the SAW system can provide an outcome of a complication. Preferably, the risk prediction model of the machine learning model can calculate a risk level of experiencing any of the PCs among 18 PCs described in Table 1. Additionally, the results of the risk calculation can be accessed by relevant health care personnel by means of a display.

Furthermore, hereby disclosed approach can be configured such that said medical report of the patient can be analysed by the trained machine learning model for providing information about whether a DNA sample of the patient should be obtained, such as genotyping and complex biomarker analysis.

In an embodiment shown in FIG. 1, the SAW system is further configured for providing a risk cut-off value. The risk cut-off value can be defined for each of 18 PCs described in Table 1. When/if the analysis of the medical report data of the patient results in a high-risk value in step 110, higher than said risk cut-off value, SAW system can suggest obtaining a sample of said patient for additional relevant genomics screening and biomarker analysis in step 120 and 122. Said sample may be a blood sample of said patient for further biomarker analysis, comprising proteomics, transcriptomic or epigenomics analysis. Alternatively, SAW system can suggest obtaining a DNA sample of said patient for additional biomarker analysis.

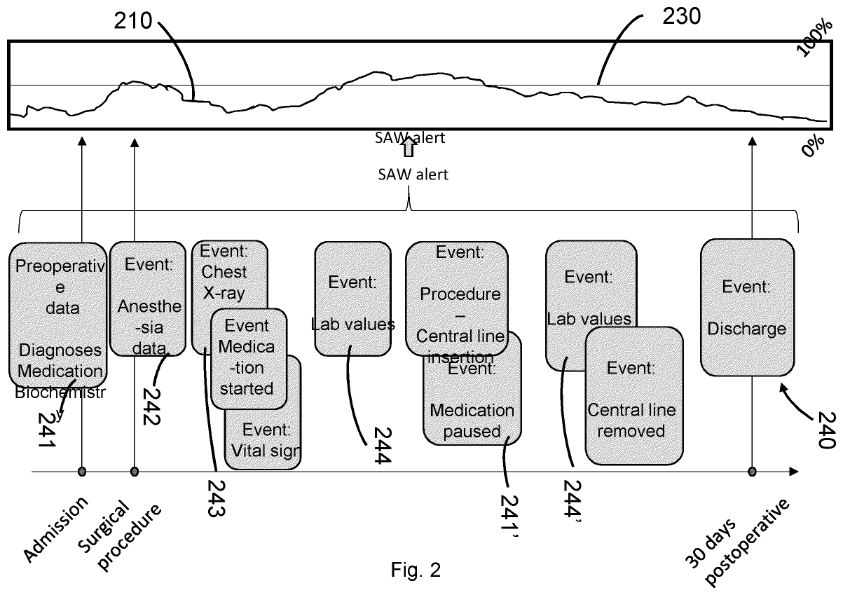

FIG. 2 is one embodiment of an overview of data flow for predicting the risk of developing deep venous thrombosis during 30 days after a surgical procedure. In this example, the risk cut-off value 230 for deep vein thrombosis is 50%. If/when the risk of having deep vein thrombosis after a surgery is calculated (in step 110 of FIG. 1) as above 50% for a patient, the risk of having deep vein thrombosis for said patient is referred as a high-risk. Thus, a high-risk prediction can be seen as a risk prediction predicted by the SAW system or by the method as described herein, wherein the risk prediction is above a previously defined risk cut-off value.

Thus, when/if the predicted risk value exceeds the risk cut-off value of 50%, a DNA-methylation analysis is suggested. Suggesting further biomarker analyses analysis is optional and can be coupled with any of the above-described 18 PCs. Predicted risk value above a predefined risk cut-off value can also be regarded as a warning for taking precautions. An advantage of hereby-disclosed real-time updated risk prediction is to allow the clinical team to counter emerging PC's. For deep venous thrombosis, this could be a personalized dose increase of thromboprophylaxis while the risk remains high, whereas the dose could be lowered when risks were in the low-risk range lower than the cut-off value.

Furthermore, SAW system can continuously monitor the medical report data of the patient after the surgery. From the date of the surgery, the outcome tracker can register a true complication outcome for a certain predetermined time for retraining the machine learning model in step 150. One example of retraining a machine learning model for predicting postoperative surgical complications can be to adapt the proposed model disclosed by Bonde et al. [1].

A 30 days overview of data flow for SAW system for predicting one of the 18 PCs; namely deep venous thrombosis, is shown in FIG. 2. As the patient moves through the pre-, intra- and post-operative phase, events 240 are generated and assessed by SAW system in real-time. Based on pre-, intra- and post-operative data, a continuous risk prediction is calculated and presented to the relevant clinical team for example in a time-risk level graph 210. In addition to the true complication outcome, the tracker can register other clinical registries such as medication changes 241, 241′, anaesthesia data 242, X-ray data 243, laboratory results 244, 244′ and so on for a certain predetermined time. Thus, from one aspect, the outcome tracker tracks the true outcome of a complication. The outcome tracker may be configured to further track medication changes, anaesthesia data, X-ray data, lab results such as ECG data (cardiac cardiograms), blood sample data, and microbiological culture data, vital signs such as pulse and blood pressure, fluid input and output data, data related to insertion and removal of drains, and intravenous access, procedural data, such as operations, physiotherapy, wound care.

The tracked and registered data can be used for retraining of the risk prediction model and thus allow for continuous updating of the machine learning model, thereby allowing the model to adapt to changes in treatment practices and patient demographics over time. This concept is referred to as a dynamic retraining.

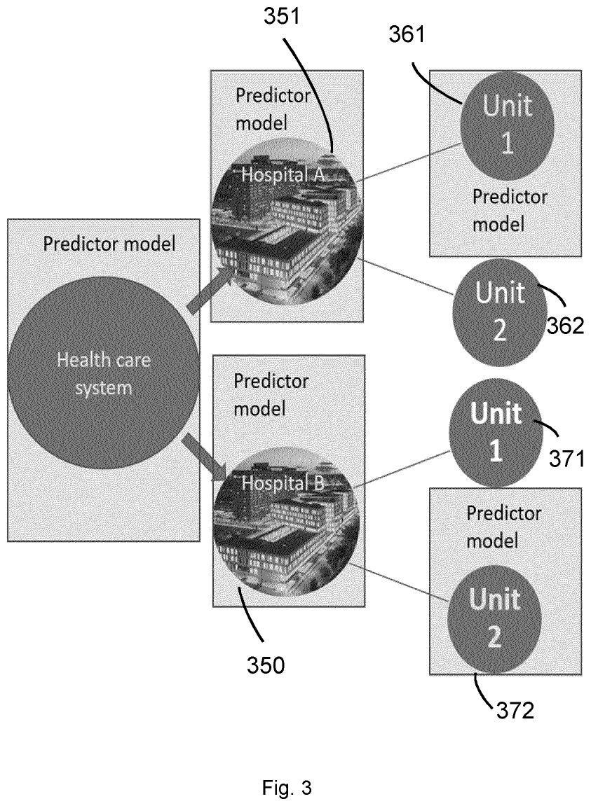

FIG. 3 shows one embodiment of an overview of data flow for training a machine learning model locally. Dynamic retraining allows for retraining on local data from individual hospitals 350, 351 and individual units 63, 362, 371, 372, thereby allowing for gradual learning of local features.

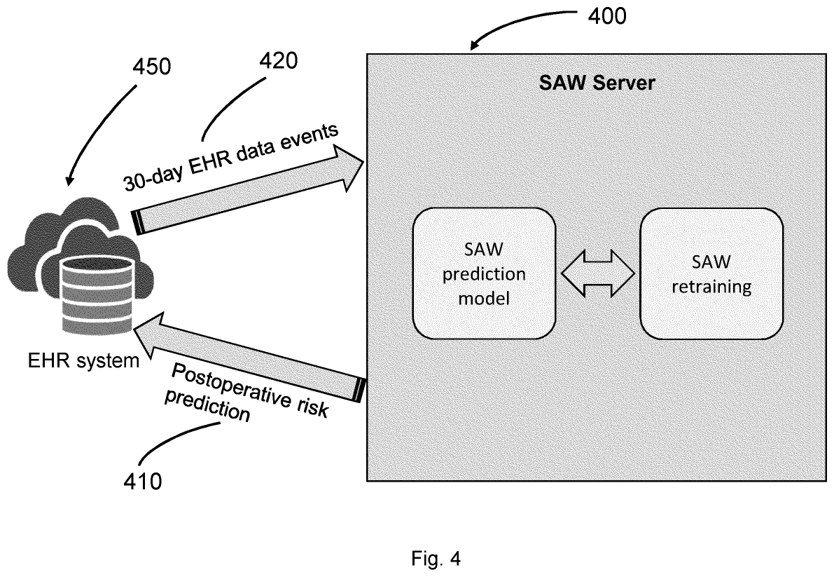

FIG. 4 shows one embodiment of an automated surgical complication tracking and reporting system overview. The system comprises an electronic health record (EHR) database interfacing with an SAW server comprising the Al model and the tracking and reporting system. On surgical case booking, EHR data events, comprising the occurrence of postoperative complications, are transferred dynamically to the server 400 for 30 days. The SAW server 400 feeds real-time dynamic risks 410, based on available EHR data events 420, back to the EHR system 450, including suggestions for augmenting biomarkers. At regular intervals, the SAW prediction model is recalibrated via the retraining system, based on the actual occurrence of postoperative complications reported to the SAW system. Thus, the risk prediction model is dynamically retrained on local surgical data based on data supplied to the tracking and reporting system, and Al model can be automatically adapted to changing surgical and/or postoperative treatments affecting the incidence of PCs.

### Reference List

[1] Bonde, A., Varadarajan, K. M., Bonde, N., Troelsen, A., Muratoglu, O. K., Malchau, H., Yang, A. D., Alam, H., & Sillesen, M. (2021). Assessing the utility of deep neural networks in predicting postoperative surgical complications: a retrospective study. The Lancet. Digital health, S2589-7500(21)00084-4. Advance online publication. https://doi.org/10.1016/S2589-7500(21)00084-4

### Items

1. A method for predicting an outcome of a complication of a patient and for further training a trained machine learning model, wherein the method comprises the steps of
receiving a medical report of a patient,
analysing said medical report for an outcome of a complication of the patient by a trained machine learning model, such as a convolutional neural network model, thereby obtaining a complication outcome,
receiving a true outcome of the complication, and
updating the trained machine learning model to become a locally trained machine learning model.

2. The method according to item 1, wherein the method comprises the steps of
receiving a second medical report of a second patient at the same location as the patient,
analysing said second medical report for a second outcome of a second complication of the second patient by the locally trained machine learning model, thereby obtaining a second complication outcome,
receiving a second true outcome of the second complication, and
updating the locally trained machine learning model to become a further locally trained machine learning model.

3. The method according to any of the preceding items, wherein the trained machine learning model and the locally trained machine learning model is updated dynamically to become the locally trained machine learning model and the further locally trained machine learning model, respectively.

4. The method according to item 3, wherein the trained machine learning model and the locally trained machine learning model is updated dynamically by updating before and/or during and/or after an operation for predicting the outcome or the second outcome.

5. The method according to any of the preceding items, wherein the step of analysing said medical report or said second medical report by the trained machine learning model provides information about whether a blood sample of the patient should be obtained, such as genotyping and complex biomarker analysis.

6. The method according to any of the preceding items, wherein the complication outcome is the outcome within a certain predetermined time, or wherein the second complication outcome is the second outcome within a certain predetermined time.

7. The method according to item 6, wherein the certain predetermined time is calculated from the step of analyzing said medical report or said second medical report or from a surgery.

8. The method according to any of the preceding items, wherein the complication is a sickness or a surgery.

9. The method according to any of the preceding items, wherein the trained machine learning model is a convolutional neural network model.

10. The method according to any of the preceding items, wherein the trained machine learning model is a long short-term neural network model.

11. The method according to any of the preceding items, wherein the trained machine learning model comprises a logistic regression model, entity embeddings and/or a random forest model.

12. The method according to any of the preceding items, wherein the medical report or the second medical report comprises pre-surgical data of the patient, such as diagnoses, medication, blood sample.

13. The method according to any of the preceding items, wherein the outcome or the second outcome of the complication comprises a risk level of the complication.

14. The method according to any of the preceding items, wherein the step of updating the trained machine learning model is preformed periodically, such as at least once a week, preferably at least once a day.

15. The method according to any of the preceding items, wherein the method comprises a step of reanalysing the medical report for an updated outcome or the second medical report for an updated second outcome of the complication of the patient by the trained machine learning model, wherein the step of reanalysing is performed after the medical report or the second medical report has been updated by an update selected from the group of
medication changes,
anaesthesia data,
X-ray data,
lab results such as ECG data (cardiac cardiograms), blood sample data, and microbiological culture data.
Vital signs such as pulse and blood pressure,
Fluid input and output data,
Data related to insertion and removal of drains, and intravenous access,
Procedural data, such as operations, physiotherapy, wound care.

16. The method according to item 15, wherein the step of reanalysing the medical report or the second medical report is performed periodically, such as at least once a week, preferably at least once a day.

17. The method according to any of the preceding items, wherein the (further) locally trained machine learning model is a first (further) locally trained machine learning model at a first location, wherein the method further comprises step of updating the trained machine learning model at a second location different from the first location to become a second (further) locally trained machine learning model.

18. The method according to item 17, wherein first (further) locally trained machine learning model and the second (further) locally trained machine learning model are compared, and wherein medical reports and true outcomes and/or second medical reports and second true outcomes of the first and second (further) locally trained machine learning models are compared for determining an optimal treatment/drug of a certain disease.

19. A data processing system comprising a processor configured to perform the steps of the method of any of the items 1-18.

20. A computer program comprising instructions, which, when the program is executed by a computer, cause the computer to carry out the steps of the method of any of the items 1-18.

1. A method for predicting an outcome of a complication of a patient and for further training a trained machine learning model, wherein the method comprises the steps of
receiving a medical report of a patient,
analysing said medical report for an outcome of a complication of the patient by a trained machine learning model, such as a convolutional neural network model, thereby obtaining a complication outcome,
receiving a true outcome of the complication, and
updating the trained machine learning model dynamically to become a locally trained machine learning model.

2. The method according to claim 1, wherein the method comprises the steps of
receiving a second medical report of a second patient at the same location as the patient,
analysing said second medical report for a second outcome of a second complication of the second patient by the locally trained machine learning model, thereby obtaining a second complication outcome,
receiving a second true outcome of the second complication, and
updating the locally trained machine learning model dynamically to become a further locally trained machine learning model.

3. The method according to any of the preceding claims, wherein the trained machine learning model and the locally trained machine learning model is updated dynamically by updating before and/or during and/or after an operation for predicting the outcome or the second outcome.

4. The method according to any of the preceding claims, wherein the step of analysing said medical report or said second medical report by the trained machine learning model provides information about whether a blood sample of the patient should be obtained, such as genotyping and complex biomarker analysis.

5. The method according any of the preceding claims, wherein the complication outcome is the outcome within a certain predetermined time, or wherein the second complication outcome is the second outcome within a certain predetermined time.

6. The method according to claim 5, wherein the certain predetermined time is calculated from the step of analyzing said medical report or said second medical report or from a surgery.

7. The method according to any of the preceding claims, wherein the complication is a sickness or a surgery.

8. The method according to any of the preceding claims, wherein the trained machine learning model is a convolutional neural network model.

9. The method according to any of the preceding claims, wherein the trained machine learning model is a long short-term neural network model.

10. The method according to any of the preceding claims, wherein the trained machine learning model comprises a logistic regression model, entity embeddings and/or a random forest model.

11. The method according to any of the preceding claims, wherein the medical report or the second medical report comprises pre-surgical data of the patient, such as diagnoses, medication, blood sample.

12. The method according to any of the preceding claims, wherein the outcome or the second outcome of the complication comprises a risk level of the complication.

13. The method according to any of the preceding claims, wherein the step of updating the trained machine learning model is preformed periodically, such as at least once a week, preferably at least once a day.

14. The method according to any of the preceding claims, wherein the method comprises a step of reanalysing the medical report for an updated outcome or the second medical report for an updated second outcome of the complication of the patient by the trained machine learning model, wherein the step of reanalysing is performed after the medical report or the second medical report has been updated by an update selected from the group of
medication changes,
anaesthesia data,
X-ray data,
lab results such as ECG data (cardiac cardiograms), blood sample data, and microbiological culture data.
Vital signs such as pulse and blood pressure,
Fluid input and output data,
Data related to insertion and removal of drains, and intravenous access,
Procedural data, such as operations, physiotherapy, wound care.

15. The method according to claim 14, wherein the step of reanalysing the medical report or the second medical report is performed periodically, such as at least once a week, preferably at least once a day.

16. The method according to any of the preceding claims, wherein the (further) locally trained machine learning model is a first (further) locally trained machine learning model at a first location, wherein the method further comprises step of updating the trained machine learning model at a second location different from the first location to become a second (further) locally trained machine learning model.

17. The method according to claim 16, wherein first (further) locally trained machine learning model and the second (further) locally trained machine learning model are compared, and wherein medical reports and true outcomes and/or second medical reports and second true outcomes of the first and second (further) locally trained machine learning models are compared for determining an optimal treatment/drug of a certain disease.

18. A data processing system comprising a processor configured to perform the steps of the method of any of the claims 1-17.

19. A computer program comprising instructions, which, when the program is executed by a computer, cause the computer to carry out the steps of the method of any of the claims 1-17.
